(19) United States
(12) Patent Application Publication
(10) Pub. No.: US 2021/0368780 A1
(43) Pub. Date: Dec. 2, 2021

(54) COMPOSITIONS FOR MAINTAINING THE VIABILITY OF LIVING AND STATIC BIOLOGICAL MATERIAL, METHODS OF MAKING AND THE USES THEREOF

(71) Applicant: NORTH GROVE INVESTMENTS, INC., Lewes, DE (US)

(72) Inventor: Joseph Fischer, Palm Beach Gardens, FL (US)

(73) Assignee: NORTH GROVE INVESTMENTS, INC., Lewes, DE (US)

(21) Appl. No.: 17/398,653

(22) Filed: Aug. 10, 2021

Related U.S. Application Data
(63) Continuation-in-part of application No. PCT/US2020/018291, filed on Feb. 14, 2020.
(60) Provisional application No. 62/805,784, filed on Feb. 14, 2019.

(51) Int. Cl. A01N 1/02

(52) U.S. Cl. CPC A01N 1/0226

(57) ABSTRACT

The present invention relates to compositions for the prolonged preservation and maintenance of living and static biological material, methods of making and uses thereof.

### CROSS-REFERENCE TO RELATED APPLICATIONS

This application is a continuation-in-part of PCT Application Serial No. PCT/US2020/018291, filed Feb. 14, 2020, which in turn, claims the benefit of priority from U.S. provisional patent application No. 62/805,784, filed on Feb. 14, 2019, each of being titled Compositions for Maintaining the Viability of Living and Static Biological Material, Methods of Making and the Uses Thereof, and the disclosure of each of which being incorporated herein by reference.

### TECHNICAL FIELD

The present invention relates to compositions for maintaining the viability of living and static biological material, particularly for preservation of organs donated for transplantation, and for fluid replacement and hydration in animals, particularly in mammals, as well as methods of making and using the same.

### BACKGROUND ART

Progress in the art of maintaining the viability of living and static biological material, and particularly for medical organ transplantation, as well as other related medical procedures, has increased the demand for all types of these biological materials. Given the stringent requirements for tissue and blood type matching, and the limited sources for donations, the supply of, for example, available hearts, livers, lungs, kidneys, and other organs, as well the supply of tissues, embryos, sperm and cells is generally substantially less than the number of patients waiting for a life-extending transplants or other necessary medical procedures from the extensive list of other biological material. Thus, there remains an ongoing need to optimize the limited supply of biological material, especially donated organs. One way that the art has sought to maximize the availability of donated organs, specifically, is by improving the preservation of organs after donation, but prior to reimplantation. The same techniques are generally used for the needed tissues, embryos, sperm and cells.

Generally, current donor organ preservation protocols seen in the prior art do not attempt to recreate an in vivo-like physiologic state for organs separated from a normal blood supply. Instead, they utilize hypothermic conditions (often below 20 degrees Celsius) and storage in an osmotically neutral, crystalloid solution, an aqueous solution of mineral salts or other water-soluble molecules; the most common crystalloid solution used today being normal saline, a solution of sodium chloride at 0.9% concentration, which is close to the concentration in human blood. Current methods for preserving viability of an organ that has been separated from its usual nutrient sources, e.g., the blood circulation of a living animal or person, depend on contacting and/or perfusing the organ with a supportive solution typically combined with reduction in organ temperature to just above the freezing point of water (i.e., just above 0° Celsius). This is intended to reduce the metabolic rate of organ tissues, thus slowing the consumption of nutrients and the production of waste products. However, the storage and transport of organs supported in this way, that is in hypothermic storage, remains very limited in time, and the reduction in the metabolic rate of organ tissues presents a number of significant problems and disadvantages. Likewise, replacing crystalloid solutions with colloid solutions, which contain larger insoluble molecules, present their own set of problems, such as those that contain albumins, and in particular those containing albumins such as BSA (“bovine serum albumin”), which may carry viruses and bacteria that are harmful to animals when introduced into the blood stream or used as a preservation medium for a donated organ destined for animal reimplantation. Still further, while products such as Hextend™, which contains hydroxyethyl starch, has shown some promise, its use in humans, who are very ill, is associated with an increased risk of death and kidney problems, and thus it is not recommended in people with known inflammatory conditions such as, inter alia, renal impairment.

It has been hypothesized that one important cause of the short storage time for reimplantation is the damage incurred during cold storage, followed by the tissue injury that occurs during warming and reperfusion with blood of the transplant recipient. Given the ongoing shortage of donated organs, there still remains a longstanding need to extend the time for storage or transport before reimplantation, wherein lowering the organ temperature to near 0° Celsius during transport, only to bring the temperature back up near body temperature before implantation is avoided, or at least kept within a much less differential degree range.

As can be readily appreciated, there remains a longstanding need in the art for compositions and methods for the improved preservation of viable organs, tissues, embryos and cells for prolonged periods away from normal circulatory support, both in vivo and in vitro. Such aforementioned compositions and methods that could also be used as a fluid replacement and hydration medium in animals, and particularly in humans, without the need for any, or at least minimal re-compounding of the composition is clearly needed. The present invention meets these significant, long-felt and yet unmet needs.

### DISCLOSURE OF INVENTION

According to one preferred embodiment, the present invention provides a composition containing a First Trace, a Second Trace and a Base, wherein the First Trace includes Arachidonic Acid, Linoleic Acid, Linolenic Acid, Myristic Acid, Oleic Acid, Palmitic Acid sodium salt, Stearic Acid, Cholesterol (non-animal source), Tween80 (i.e., Polysorbate 80) (non-animal source), DL-a-Tocopherol and Vitamin A acetate (i.e., Retinol), wherein the Second Trace includes D-Biotin, L-Cysteine hydrochloride monohydrate (non-animal source), Folic Acid, Reduced Glutathione, Riboflavin, Thiamine hydrochloride and Vitamin B12, wherein the Base includes L-Arginine hydrochloride, L-Aspartic Acid, Adenosine, L-Ascorbic Acid, Calcium Chloride anhydrous, Choline Bitartrate, Dextran-40, Glycine, L-Glutamic Acid, L-Glutamine, D-Glucose anhydrous, L-Histidine hydrochloride monohydrate, L-Isoleucine, L-Leucine, L-Lysine hydrochloride, Magnesium Sulfate anhydrous, D-Mannose, L-Proline, L-Phenylalanine, Potassium Phosphate monobasic, Poloxamer 188/Pluronic F-68, Sodium Phosphate monobasic monohydrate, Sodium Gluconate, L-Threonine, L-Tryptophan and L-Valine, and further wherein the composition includes L-Cystine and L-Tyrosine.

According to another preferred embodiment, the composition of the present invention is a single solution.

According to another preferred embodiment, the composition of the present invention is a single solution compounded from one aqueous component and two powder components.

According to another preferred embodiment, the composition of the present invention is compounded into anyone of the following forms: (1) an aqueous solution, (2) a powder, (3) (4) a cream, (5) an ointment, (6) a paste, or (7) a gel.

According to another preferred embodiment, the composition is essentially free of all human and non-human animal proteins, growth factors and hormones.

According to another preferred embodiment, the composition contains nanoparticles or liposome components.

According to another preferred embodiment, the pH of the composition is preferably kept at a pH of from about 7.1 to about 7.3.

According to another preferred embodiment, the osmolality of the composition is within a range of from about 320 mM/Kg to about 430 mM/Kg.

According to another preferred embodiment, a method of preparing the composition includes the steps of combining the First Trace, the Second Trace and the Base.

According to yet another preferred embodiment, a method of preserving a mammalian organ, ex vivo, includes the steps of contacting or perfusing the mammalian organ with an effective amount of a composition of the present invention.

According to yet another preferred embodiment, a method of providing perfusion support for organs or tissues acutely deprived of normal blood circulation includes the step of administrating a composition of the present invention.

According to yet another preferred embodiment, a method of treating a human or non-human animal in need of fluid replacement includes the step of administrating a composition of the present invention.

According to yet another preferred embodiment, a method of protecting living or static biological material includes the step of administrating a composition of the present invention.

According to yet another preferred embodiment, a method of preserving an organ includes the step of administrating a composition of the present invention.

According to yet another preferred embodiment, a method of repairing an anatomical area damaged by disease or accident includes administrating a composition of the present invention.

According to another preferred embodiment, a composition includes Arachidonic Acid, Linoleic Acid, Linolenic Acid, Myristic Acid, Oleic Acid, Palmitic Acid sodium salt, Stearic Acid, Cholesterol (non-animal source), Tween80 (i.e., Polysorbate 80) (non-animal source), DL-a-Tocopherol, Vitamin A acetate (i.e., Retinol), D-Biotin, L-Cysteine hydrochloride monohydrate (non-animal source), Folic Acid, Reduced Glutathione, Riboflavin, Thiamine hydrochloride, Vitamin B12, L-Arginine hydrochloride, L-Aspartic Acid, Adenosine, L-Ascorbic Acid, Calcium Chloride anhydrous, Choline Bitartrate, Dextran-40, Glycine, L-Glutamic Acid, L-Glutamine, D-Glucose anhydrous, L-Histidine hydrochloride monohydrate, L-Isoleucine, L-Leucine, L-Lysine hydrochloride, Magnesium Sulfate anhydrous, D-Mannose, L-Proline, L-Phenylalanine, Potassium Phosphate monobasic, Poloxamer 188/Pluronic F-68, Sodium Phosphate monobasic monohydrate, Sodium Gluconate, L-Threonine, L-Tryptophan, L-Valine, L-Cystine and L-Tyrosine.

Further detailed description of preferred embodiments of the invention is provided below, and with representative examples, however these preferred embodiments and examples do not limit the scope of the invention in any way.

### BRIEF DESCRIPTION OF THE DRAWINGS

FIG. 1 is a graph depicting the re-beating time in seconds after a heart graft re-transplant in mice from a cervical model heart transplant study as set forth in the examples;

FIG. 2 is a graph depicting a circulatory relationship between hypothermic preservation at 12 h and 24 h as set forth in the examples; and

FIG. 3 is a graph depicting an evaluation of cardiac function after transplantation as described in the examples.

### BEST MODE FOR CARRYING OUT THE INVENTION

The present invention provides compositions for maintaining the viability of living and static biological material in vivo, ex vivo and/or in vitro, as well as methods of making and using these compositions. Broadly, the inventive compositions are formulated to include supportive and/or preservative nutrients and other substances for maintaining the health and viability of living and static biological material in vivo and ex vivo at non-hypothermic temperature ranges (for example, but not limited to, temperatures ranging from about 20° Celsius to about 37° Celsius). By maintaining the health and viability of living and static biological material both in vivo and ex vivo at non-hypothermic temperature ranges, the compositions of the present invention are surprisingly advantageous because it is unnecessary to reduce, slow down or stop cellular metabolism when using these inventive compositions. The present invention also avoids all the problems associated with cryopreservation of living and static biological material.

Broadly, and in most preferred aspects of the invention, the inventive compositions include combinations of various components, including but not limited to those selected from among, amino acids, various fatty acids, salts, sugars, trace elements, vitamins, certain carbohydrates, surfactants, emulsifiers, and volume expanders. In preferred embodiments, the compositions can be further supplemented with combinations of ingredients which can include, but are not limited to, buffers, anti-inflammatories, and antioxidants, which are dissolved or dispersed in an aqueous medium or other contemplated form. The inventive compositions also preferably contain many nutrient and mineral factors at concentrations analogous to those found in blood, serum, plasma, and/or normal body tissues.

Reference will now be made in detail to various aspects of the invention and embodiments. The following language and descriptions of certain preferred embodiments of the present invention are provided to further an understanding of the principles of the present invention. However, it will be understood that no limitations of the present invention are intended, and that further alterations, modifications, and applications of the principles of the present invention are also included.

Moreover, unless otherwise defined, all numbers expressing quantities of ingredients, properties such as molecular weight, reaction conditions, and so forth used in the specification are to be understood as being modified in all instances by the term “about.” Accordingly, unless indicated to the contrary, the numerical parameters set forth in the specification are approximations that may vary depending upon the desired and intended properties.

The compositions of the present invention can be used to maintain the viability of any type of biological material. As used herein, the term “biological material’ is very broadly intended to include anything that is part of a living organism, or that was derived or obtained from a living organism, including any human or non-human animal or other type of living organism. Some representative, non-limiting examples of biological material include, but are not limited to, any type of organ, cell, tissue, or any other type of biological material such as, for example, embryo or sperm. The biological material may be in any state or condition, including but not limited to a static or non-static condition. The present invention also contemplates that the biological material may be obtained directly from a non-human animal, or a human or other living organism, without any genetic modification, or the biological material may be altered or modified in some manner, e.g., genetically modified or otherwise altered by some type of intervention (for example, altered by CRISPR gene editing). These are merely given as non-limiting examples of biological material and these examples are not intended to limit the scope of the present invention in any way.

The term “aqueous solution”, as used herein, refers to a solution in which the solvent is water and includes but is not limited to buffers with inorganic salts such as sodium or potassium phosphates, sodium, potassium or calcium chlorides, sodium or potassium acetate as well as organic and inorganic acids and bases such as sodium or potassium hydroxide, acetic acid etc.

The term “composition” as used herein is intended to refer, but is not limited to, any formulation, mixture or other combination of constituent components or ingredients that may be used for one or more beneficial purposes, for example but not limited to pharmaceutical and/or medical uses in human and/or non-human animal subjects.

The present invention contemplates compositions of the present invention, including but not limited to the D2 liposome complex, wherein the compositions include a First Trace, a Second Trace and a Base, and wherein these compositions can be manufactured using any suitable process or processes, such as, and merely by way of example, milling and mixing and/or dissolving in soluble based mediums.

The term, “organ” as used herein encompasses, but is not limited to, both solid organs, e.g., kidney, heart, liver, lung, as well as functional parts of organs, e.g., segments of skin, sections of artery, transplantable lobes of a liver, kidney, lung, and the like. The term, “tissue” refers herein to viable cellular materials in an aggregate form, e.g., small portions of an organ, as well as dispersed cells, e.g., cells dispersed, isolated and/or grown from heart muscle, liver or kidney, including bone marrow cells and progeny cells, blood born stem cells and progeny, and the various other art-known blood elements, unless otherwise specified. The term “embryo” as used herein refers to early developmental stage multicellular diploid eukaryotic organisms. The term “sperm” as used herein refers to one of the minute, usually actively motile gametes in semen, which serve to fertilize an ovum. And the term “cell” as used herein means the smallest structural and functional unit of an organism, typically microscopic and consisting of cytoplasm and a nucleus enclosed in a membrane.

As used herein, the term “substantially” or “essentially” shall be understood to be definite terms that broadly refer to a degree that is, to a significant extent, close to absolute, or essentially absolute. For example, the term “essentially free” shall be understood to be a definite term that broadly refers to a degree which is close to being absolutely free. Also, by way of non-limiting example, the term “substantially complete” shall refer to a degree of completeness that is at least about ninety percent or more complete, or that is, to a significant extent, essentially 100 percent complete.

The term “wt. %” or “weight %” refers to a concentration by weight of a component in the entire composition.

Further, the use of singular terms for convenience in description is in no way intended to be so limiting. Thus, simply for illustration, reference to a composition comprising “a nanoparticle” includes reference to one or more of such nanoparticles, e.g., to a preparation with sufficient nanoparticles for the intended purpose, unless otherwise stated.

### Representative Compositions of the Present Invention

One preferred embodiment of the present invention is directed to a liposome complex containing a “D2 Formulation” as described in further detail herein. This is also sometimes interchangeably referred to herein as the “D2 liposome complex.” As used herein, the term “D2 liposome complex” is intended to refer to a “liposome complex” which preferably encompasses, but is not limited to, a complex that includes a “nanoparticle” lipid emulsion or liposome component.

The term, “nanoparticle” as employed herein is defined as, but is not limited to, a two-layer emulsion particle, preferably with a lipophilic outer layer and a hydrophilic core, in a size (mean diameter) ranging from about 100 nm to about 300 nm, and more preferably in a size ranging from about 100 nm to about 200 nm.

A “nanoparticle” lipid emulsion or liposome component, as used herein, is intended to refer to a lipid emulsion or liposome component that includes a lipophilic outer layer and a hydrophilic inner core. This includes a lipid and/or sterol outer membrane, and essential fatty acids, and a hydrophilic inner core. The hydrophilic inner core includes essential materials such as protein-derived growth factors and optionally, additional substances, such as ATP, and the like. In certain optional embodiments, this inner core can be include or be replaced with a suitable oxygen carrier, e.g., a heme protein or solution or suspension of heme proteins, including, for example, a naturally derived heme, a recombinant heme optionally mutated or chemically modified to have an oxygen saturation curve effective to transport and deliver oxygen and remove carbon dioxide in a harvested organ or tissue, and/or an artificial water soluble heme, to name but a few types of oxygen carriers.

Advantageously, the inventive compositions contain no human or non-human animal sera or undefined proteins.

Without meaning to be bound by any theory or hypothesis as to how the inventive compositions might operate, upon contact with cell membranes of treated cells, the hydrophobic outer layer fuses with the cell membrane, allowing the hydrophilic core of the inventive nanoparticle to be taken up by those cells into the cytoplasm, thereby delivering viability-enhancing supplemental energy compounds and essential growth factors. Also the elevated osmolality, relative to the osmolality of normal body fluids, operates to mitigate cellular swelling, and to facilitate the preservation of vascular cellular integrity.

In certain alternate embodiments, the inventive compositions comprise a base nutritive medium which preferably includes, in physiologically suitable concentrations, salts, water soluble vitamins, amino acids and nucleotides. These include, simply by way of example, and without limitation, adenosine and its phosphates, uridine and its phosphate, other nucleotides and deoxynucleotides; B vitamins, e.g., B1, B2, B6, B12, biotin, inositol, choline, folate, and the like; vitamin coenzymes and co-factors, e.g., nicotinamide and flavin adenine dinucleotides, and their respective phosphates, coenzyme A and the like; various physiological salts and trace minerals, e.g., salts of sodium, potassium, magnesium, calcium, copper, zinc and iron; the essential amino acids, although all twenty naturally-occurring amino acids, and/or derivatives thereof, are optionally included. The base nutritive medium also includes, e.g. pH buffers, such as phosphate buffers and N-2-hydroxyethylpiperazine-N′-2-ethanesulfonic acid (“HEPES”) buffer; simple sugars, e.g., glucose; osmotic enhancers, such as any suitable dextran, mannose and the like; as well as optional miscellaneous components, such as, allopurinol, chondroitin, carboxylase, physiological organic acids, e.g., pyruvate, and optionally, a nutritive extract from natural sources, e.g., a yeast vitamin extract.

In another alternative embodiment, Vitamin C is optionally included in physiological or higher than physiological concentrations.

The compositions of the present invention also preferably include a lipid-aqueous emulsion comprising liposomes or nanoscale particles with a lipophilic outer layer and a hydrophilic core. Generally, this includes lipophilic components able to form and stabilize the outer, lipophilic layer, including, for example, Cholesterol, Phosphatidylcholine, Vitamin E, Cod Liver Oil, etc. Additional components preferably include lipid-based energy sources, including physiologically compatible amounts of free fatty acids such as Linoleic, Linolenic, Oleic acid and their functional equivalents.

In another alternate embodiment, the lipid-aqueous emulsion also preferably includes hydrophilic supportive components. Further supportive components can include, for example, intercellular messengers such as prostaglandins, e.g., prostaglandin E1. Preferably, physiologically compatible surfactants and detergents are also included, e.g., one or more water-soluble surfactants, preferably an amphiphilic block copolymer with a molecular weight of several thousand Daltons, such as a polyethylene oxide-polypropylene oxide block copolymer surfactant (e.g., Pluronic F-68; from BASF) and/or nonionic surfactants. Suitable nonionic surfactants include, e.g., polyoxyethylene derivatives of sorbitol esters, e.g., polyoxyethylene sorbitan monooleate surfactants that are commercially available as TWEEN (Atlas Chemical Co.). TWEEN80^{†} is particularly preferred. The core portion of the compositions of the invention preferably may not include a pharmaceutically significant quantity of a phosphatidic acid or sugar, or a lysophosphatidic acid or sugar.

In other certain embodiments, the present invention is directed to nanoparticle compositions for maintaining the viability of living and static biological material when such are separated from normal physiological supports. Compositions containing the nanoparticle compositions and methods of preserving organs such as kidneys, both in vivo and ex vivo, are also disclosed.

### Further Description of One Preferred Embodiment: The “D2 Formulation”

In one preferred embodiment, the present invention contemplates a chemical solution (hereinafter referred to as the “D2 Formulation”) having various constituent chemical components. More particularly, in a preferred embodiment, the invention contemplates a “D2 Formulation” which preferably includes a First Trace solution, a Second Trace powder and a Base powder. For example, a D2 Formulation may include the following components:

1. The First Trace (dissolved to form an aqueous solution)

2. The Second Trace (ground to a homogenous powder form)

3. The Base (milled to a homogenous powder form)

4. Sodium Hydroxide (NaOH)

5. L-Cystine (C₆H₁₂N₂O₄S₂)

6. L-Tyrosine (C₉H₁₁NO₃)

According to this preferred embodiment, the First Trace solution includes the following preferred ingredients (shown below in Table 1). The present invention also contemplates various possible alternate ingredients and non-limiting examples of these various possible alternate ingredients are also listed and shown below in Table 1. It is to be understood that these non-limiting examples of alternate ingredients are provided by way of illustration only, and do not limit the scope of the present invention in any way.

TABLE 1
The First Trace solution

| | Non-limiting examples of various possible |
|---|---|
| Preferred ingredients | alternate ingredients |
| Ethanol, Absolute | Glycerol, propylene glycol, polyethylene |
| | glycol-400 (PEG-400) |
| Arachidonic Acid | Arachidic acid |
| Linoleic Acid | α-Linoleic acid, LC n-3 PUFA (long chain n-3 |
| | poly-unsaturated fatty acids), gamma- |
| | linolenic acid (GLA), dihomo-gamma-linolenic |
| | acid (DGLA) |
| Linolenic Acid | α-Linolenic acid and [symbol] -Linolenic acid |
| Cholesterol | Pure DOPCC, Pure DPCC (1,2-dipalmitoyl-sn- |
| (non-animal source) | glycero-3-phosphatidylcholine) |
| Tween 80 | Tween 20, Span 80 (both non-animal source) |
| (Polysorbate 80) | |
| (non-animal source) | |
| DL-α-Tocopherol | Ascorbic acid, Ascorbyl palmitate |
| Vitamin A acetate | Non-vitamin A carotenoids |
| (Retinol) | |

Moreover, according to this preferred embodiment, the Second Trace powder includes the following preferred ingredients (shown below in Table 2). The present invention also contemplates various possible alternate ingredients and non-limiting examples of these various possible alternate ingredients are also listed and shown below in Table 2. It is to be understood that these non-limiting examples of alternate ingredients are provided by way of illustration only, and do not limit the scope of the present invention in any way.

TABLE 2
Second Trace powder

| | Non-limiting examples of various |
|---|---|
| Preferred ingredients | possible alternate ingredients |
| D-Biotin | D-Pantothenic acid |
| L-Cysteine hydrochloride | N-Acetyl cysteine (NAC) |
| monohydrate | |
| (non-animal source) | |
| Reduced Glutathione | Cysteine, glycine, and glutamic acid |
| | (in combination) |
| Thiamine hydrochloride | Thiamine, Thiamine mononitrae |
| Vitamin B12 | Methylcobalamin |

The BASE powder
Chemical
L-Arginine hydrochloride
L-Aspartic Acid
Adenosine
L-Ascorbic Acid
Calcium Chloride anhydrous
Choline Bitartrate
Dextran-40
Glycine
L-Glutamic Acid
L-Glutamine
D-Glucose anhydrous
L-Histidine hydrochloride monohydrate
L-Isoleucine
L-Leucine
L-Lysine hydrochloride
Magnesium Sulfate anhydrous
D-Mannose
L-Proline
L-Phenylalanine
Potassium Phosphate monobasic
Poloxamer 188/Pluronic F-68
Sodium Phosphate monobasic monohydrate
Sodium Gluconate
L-Threonine
L-Tryptophan
L-Valine

The compositions of the present invention, including but not limited to the D2 Formulation, may be optionally combined with suitable oxygen carriers for enhanced maintenance of tissue and cell viability.

### Representative Methods and Processes of Making the Compositions

The inventive compositions are generally preferably produced by preparing specific combinations of the necessary ingredients which are used as building blocks for the final product. The present invention contemplates preparation of the compositions of the present invention, including but not limited to the D2 liposome complex (non-animal source), wherein the compositions include a First Trace, a Second Trace and a Base.

The compositions of the present invention can be prepared or manufactured using any suitable process or processes. Certain representative examples of methods of preparing the compositions of the present invention are described herein, and these examples do not limit the scope of the invention in any way.

In one representative approach, when a liposome complex is prepared (such as the D2 liposome complex described herein), a microfluidizer or similar such apparatus is utilized, under conditions effective to provide a finely divided emulsion, e.g., a nanoparticle-scale emulsion, with the nanoparticles having a preferred mean diameter of from about 100 nm to about 200 nm. The resulting nanoparticle-scale emulsion composition provides various trace nutrients, and other components, and provides all of the surprising and unexpected advantages as described herein.

Use of Microfluidization (e.g., by a Microfluidizer or Similar Such Apparatus)

The present invention contemplates the use of “nnicrofluidization” involving techniques of high pressure homogenization, at pressures at or above 5000 psi. In preferred embodiments, this “nnicrofluidization” process can be used to create liposomes or nanoparticles with a uniform size distribution of a mean diameter of preferably from about 100 nm to about 300 nm and more preferably from about 100 nm to about 200 nm. In alternative aspects of the invention, the particles have a mean diameter of less than 200 nm. In addition to microfluidization, other standard emulsification methods can be optionally employed, e.g., sonication, valve homogenization and blade stirring, etc. Desirably, a water-soluble surfactant, preferably an amphiphilic block copolymer with a molecular weight of several thousand Daltons, such as a polypropylene oxide-polyethylene oxide block copolymer surfactant (e.g., Pluronic F-68 that is commercially available from BASF) and/or TWEEN80 (non-animal source), is added to the aqueous solution in order to stabilize the coated particles against aggregation as they form. The surfactant also serves to enhance the effect of (ultra)sonication, if that method is employed. An example of a preferred apparatus for microfluidization is the Microfluidizer No. HC5000V from Microfluidics Corp. (Newton, Mass.) using compressed air supplied by an encapsulated air compressor, e.g., No. ES-6 from Sullair Solutions (Michigan City, Ind.). The above-described apparatus employs high pressure and high shear homogenization to treat and emulsify the Premix-II composition and provide the nanoparticles within the desired size range.

The compositions of the present invention, including for example a liposome complex, can be prepared by high pressure homogenization using a microfluidizer. In preferred embodiments, the components are added to the microfluidizer reservoir in a continuous fashion and forced through the specially designed cavitation or interaction chamber, where high shear stress and cavitation forces formed a highly divided emulsion. Through multiple cycles, the mean droplet or liposome size, distribution, and combination of ingredients yield the desired end product, e.g., the preferred nanoparticles.

Components of the compositions of the invention can be used in any desired or suitable amounts or quantities to prepare a desired batch volume, for example, a total batch volume or end volume after all the components have been processed into a microscale or nanoscale emulsion.

The compositions of the invention are also prepared by dissolving or dispersing components in an order that is effective to achieve a uniform and clear aqueous composition, while avoiding undesirable reactions or to the formation of insoluble complexes.

The present invention also contemplates that the compositions of the invention can be packaged in containers that minimize, reduce, prevent or essentially eliminate exposure to light, thus reducing or essentially eliminating photo-oxidation of the components of the compositions.

The present invention also contemplates that the methods and processes described herein can be readily scaled up or down for smaller or larger batch sizes, depending on need.

All chemicals used in the preparation of the inventive composition are of substantial purity and available from numerous commercial suppliers of biochemicals. Preferably, these are of USP grade or equivalent. The artisan will appreciate that the employed chemicals are optionally substituted by substantially equivalent chemicals demonstrating the same purity and activity.

According to one preferred representative method, as described herein, the D2 Formulation can be safely and reliably prepared. And, in this example, the D2 Formulation includes the First Trace; the Second Trace; the Base; L-Cystine; and L-Tyrosine.

Sample Preparation of the First Trace (i.e., One Aggregate Component of a Preferred Final Composition of the Present Invention)

According to one non-limiting example of preparing a composition of the present invention, the first step comprises preparation of the First Trace, also referred to herein as the Trace 1 solution. In this example, each of a number of constituent First Trace ingredients or chemicals are weighed and dissolved one at a time in a suitable amount of a solvent, to form the Trace 1 solution. For example, each constituent First Trace ingredient or chemical is dissolved one at a time in about ten (10) to about thirty-five (35) mL of solvent. Preferably the solvent is ethanol, and more preferably the solvent is absolute ethanol, although other similar and suitable ethanol equivalents could be used. In this example, the First Trace ingredients or chemicals comprise Arachidonic Acid, Linoleic Acid, Linolenic Acid, Myristic Acid, Oleic Acid, Palmitic Acid Sodium, Stearic Acid, Cholesterol (non-animal source), Tween 80 (Polysorbate 80) (non-animal source), DL-a-Tocopherol and Vitamin A acetate (Retinol). It has been found that the DL-a-Tocopherol and Vitamin A acetate (Retinol) help protect the integrity of the liposome complex. Then the Trace 1 solution comprising the First Trace ingredients or chemicals is brought to a final volume preferably with deionized (DI) water, though in alternate embodiments, either distilled or sterile water could be used. The constituent chemicals can be measured using any suitable instrument, for example, with a pipette. The resulting Trace 1 solution can be stored in sterile conditions in any suitable container and is preferably stored at about negative twenty degrees Celsius (−20° C.) to about negative five degrees Celsius (−5° C.). As further described herein, with reference to Example 1, these methods can be utilized for the safe and reliable preparation of the First Trace solution. Any suitable lot size can be prepared, and a 100 liter lot size is just one example.

Sample Preparation of the Second Trace (i.e., Another Aggregate Component of a Preferred Final Composition of the Present Invention)

Continuing with the same example described above, and according to a preferred embodiment, after preparation of the First Trace, the next step involves preparation of the Second Trace, wherein the Second Trace ingredients includes D-Biotin, L-Cysteine hydrochloride monohydrate (non-animal source), Folic Acid, Reduced Glutathione, Riboflavin, Thiamine hydrochloride and Vitamin B12. In this example, the ingredients for the Second Trace are preferably ground in a mortar and pestle until it reaches a fine homogenous powder. One example of the Second Trace is described in further detail herein in Example 1. Any suitable lot size, for example a lot size of 1000 L (liters) of powder, can be made. The Second Trace powder can be stored in any suitable container and at any suitable temperature, but is preferably stored at a temperature of from about two degrees Celsius (2° C.) to about eight degrees Celsius 8° C.

Sample Preparation of the Base (i.e., Another Aggregate Component of a Preferred Final Composition of the Present Invention)

Continuing with the same example described above, and according to a preferred embodiment, and after preparation of the Second Trace, the next step involves preparation of the Base, wherein the Base ingredients includes L-Arginine hydrochloride, L-Aspartic Acid, Adenosine, L-Ascorbic Acid, Calcium Chloride anhydrous, Choline Bitartrate, Dextran-40, Glycine, L-Glutamic Acid, L-Glutamine, D-Glucose anhydrous, L-Histidine hydrochloride monohydrate, L-Isoleucine, L-Leucine, L-Lysine hydrochloride, Magnesium Sulfate anhydrous, D-Mannose, L-Proline, L-Phenylalanine, Potassium Phosphate monobasic, Poloxamer 188/Pluronic F-68 Sodium Phosphate monobasic monohydrate, Sodium Gluconate, L-Threonine, L-Tryptophan and L-Valine, and further wherein the final composition includes L-Cystine and L-Tyrosine. It is preferred that the Base is formed in powder form. Also, it is possible to use other Dextran as an alternative to the preferred Dextran-40 as listed above in the preferred embodiment.

The ingredients of the Base (listed above) are preferably mixed, and it is preferred that the ingredients of the base are milled in short intervals of about one (1) hour to prevent sticking to the mill. Then the mill is preferably allowed to sit for about thirty (30) minutes between intervals, to help avoid over-heating during the process. In this example, a lot size of 500 liters (500 L) is prepared. One example of the Base is further described in detail in Example 1. Every lot of the Base is formulated accurately and in accordance with all current standard operating procedures pertaining to powder media production. All lots as well undergo rigorous milling and process quality testing. Particle size testing is also performed. The Base is preferably stored at a temperature of from about two degrees Celsius (2° C.) to about eight degrees Celsius (8° C.).

Sample Preparation of a Preferred Final Composition of the Present Invention, Utilizing the Sample First Trace, Second Trace, and the Base

After preparation of the First Trace, Second Trace and the Base, and continuing with this example, using the ingredients as described above, the final composition of the present invention is prepared according to the following representative procedure. The following components are combined to prepare the final “D2 Formulation”:
The First Trace$
The Second Trace
The Base $
Sodium Hydroxide (NaOH)
L-Cystine; and
L-Tyrosine.

In this non-limiting example, a 100 liter (100 L) lot size is prepared and is preferably stored at a temperature of from about two degrees Celsius (2° C.) to about eight degrees Celsius (8° C.). Any suitable lot size and storage temperature can be used. During the preparation of the final “D2 Formulation,” it is preferred that the following steps are performed. Approximately 1 to 2 grams (1 to 2 g) of Sodium Hydroxide is dissolved in about 100 mL water. Then L-Cystine and L-Tyrosine are dissolved in the Sodium Hydroxide solution. More Sodium Hydroxide can be added if needed to solubilize the L-Cystine and L-Tyrosine. The Sodium Hydroxide solution [containing the L-Cystine and L-Tyrosine dissolved therein] is then added to the vat containing: (i) the First Trace, (ii) the Second Trace, and (iii) the Base. Examples of specific amounts of the First Trace, Second Trace and the Base are provided by way of illustration in Example 1.

In other embodiments, the present invention also contemplates that many commercially available cell or tissue culture media products that are free of undefined proteins or animal sera, can also be utilized for preparing the compositions, provided that such media are compatible with the specific requirements of the inventive compositions herein. Examples of commercially available cell or tissue culture media products include, but are not limited to, Dulbecco's Modified Eagle's medium (DMEM) and also modified DMEM.

For example, and for illustration purposes only, a composition preferably has, in addition to the basic cellular nutrient media described herein, the following features and elements: energy substrates to replenish the intracellular ATP energy pool, and to provide for aerobic metabolism during the perfusion and preservation process; and one or more antioxidants and/or xanthine oxidase inhibitors to mitigate reperfusion injury due to the presence and/or formation of free oxygen radicals.

### Other Preferred Embodiments: Including Other Agents in the Compositions

In certain alternate embodiments, the inventive compositions further include one or more safe and effective therapeutic agents. Any suitable type of therapeutic agent can be included, as deemed necessary or appropriate by a healthcare professional. Such examples of therapeutic agents include, but are not limited to, one or more antimicrobial agents, such as antibiotics, antibacterials, specific antibodies and/or other art-known agents for controlling microbial contamination in organs, tissues, embryos and/or cells. Examples of therapeutic agents are referenced by Goodman & Gilman's, THE PHARMACOLOGICAL BASIS OF THERAPEUTICS, 10th Edition, McGraw Hill, incorporated by reference herein in its entirety.

In certain additional alternate embodiments, the inventive compositions further include one or more of the following: an anticoagulant, a thrombolytic, and an antiplatelet drug agent to prevent clotting or fibrin formation during organ preparation, storage, transport and transplantation, e.g., heparin and related glycosaminoglycans; dicumarol, phenprocoumon, acenocoumarol, ethyl biscoumacetate, indandione, and derivatives thereof, aspirin and dipyridamole, and the like.

Non-steroidal anti-inflammatory agents can also be optionally included in certain alternate embodiments, e.g., where it is believed that inflammatory processes are etiologic in shorting the useful storage life of an organ, tissue, embryos or cells intended for transportation for transplant and/or research and development or other analysis, such as pathological examination. All of the foregoing agents are set forth in greater detail by Goodman & Gilman's, Id., as incorporated herein by reference. The amount of these agents or compounds included is described as an amount that is effective to achieve the desired therapeutic result. It will vary somewhat depending on the composition selected and the needs of the artisan. By way of example, one or more optional agents may be present in amounts ranging from about 0.01 to about 10% of the final solution.

### Representative Advantages of the Compositions of the Present Invention

As described herein, it is to be understood that “the compositions of the present invention,” as described herein, is intended to include, but is not limited to, the D2 liposome complex. The compositions of the present invention have a number of significant and unexpected advantages and unintended uses. Some of these representative advantages and intended uses are described herein below, but it is to be understood that these are described merely for illustration purposes and does not limit in any way all of the advantages and intended uses of the composition of the present invention.

Free or Essentially Free of all Human and Non-Human Animal Proteins

Unlike conventional compositions, which often contain one or more human and non-human animal proteins, growth factors and/or hormones, the compositions of the present invention are free or essentially free of all human and non-human animal proteins, growth factors and/or hormones. This reduces or preferably eliminates concerns about potential tumors or cancer caused by the presence of growth factors. The compositions of the present invention therefore have an unexpectedly and significantly improved safety profile, as compared to conventional compositions of the prior art.

The compositions of the present invention are free or essentially free of any viruses or other pathogens. The compositions of the present invention thus have a significantly improved safety profile and are much safer compared to conventional “blood substitutes,” hydration mediums, and conventional organ preservation media of the prior art.

Enhanced Viability of Organs, Tissues, Embryos and Cells

In accordance with the present invention, it has been discovered that the novel compositions and formulations of the present invention can be used as an organ preservation medium to preserve one or more organs without having to utilize hypothermic conditions for preserving the one or more organs. The novel compositions and formulations of the present invention thus do not reduce the metabolic rate of living or static biological material, such as, by way of example, organs, tissues, embryos, sperm and cells, and this unexpectedly and significantly enhances the viability of such living and static biological material.

Enhanced Oxygenation of Biological Material

The compositions of the present invention also provide enhanced oxygenation of living biological material, such as, by way of example, organs, tissues, embryos, sperm and cells, and improvements in viability and longevity of such biological material during storage and transportation. This reduces or preferably eliminates concerns about necrosis of organs and tissues, and also helps reduce or prevent apoptosis (programmed cell death) of embryos, sperm and cells or organ tissues during storage.

After administration animals, it has also been discovered that the compositions of the present invention provide additional unexpected benefits because they have been found to pass the blood-brain barrier.

### Other Representative Advantages and Benefits

The compositions of the present invention also provide additional benefits since certain components of the compositions may also have immune-enhancing, immunostimulant, antioxidant, anti-inflammatory, nutraceutical and/or other benefits. Also, as described herein, one or more other therapeutic agents (for example, but not limited to, one or more antibiotics) may also be included in the compositions.

Early test data indicates that the compositions of the present invention have a wide range of surprising and unexpected benefits, and that they have a very high degree of safety and are very effective at enhancing the viability of living biological material, such as, by way of example, organs, tissues, embryos, sperm and cells.

Early test data shows that the compositions of the present invention are safe and effective when administered as an intravenous fluid in dogs or other non-human animals for hydration and fluid replacement, and it hypothesized that the same will be shown to be safe and effective for humans after proper testing has been completed.

### Quality Control of the Compositions of the Present Invention

It is also contemplated that quality control studies of the compositions of the present invention, for example but not limited to the “D2 formulation” can also be performed (see Example 1 for sample results from quality control studies). By performing quality control studies, several criteria of the compositions can be evaluated including, but not limited to, appearance, sterility, sterile filtration, pH, osmolality, endotoxin levels, mycoplasma levels, and expiration (i.e., shelf life) of the compositions. Sterile filtration can be performed with any suitable size filter, for example, a 0.22 micron filter.

It has been found that the compositions of the present invention can be formulated to have a very long shelf-life or storage, for example, but not limited to, up and to twenty-four (24) months of storage and under sterile and temperature and humidity-controlled conditions to meet all regulatory specifications and requirements e.g. for commercial purposes.

Representative results of such quality control studies are described and shown in Example 1. The results shown in Example 1 are simply for illustration purposes only, and do not limit the scope of the invention in any way.

It is also contemplated that the compositions of the present invention can be reliably and efficiently formulated, prepared or manufactured, as well as packaged and tested, to comply with any regulatory requirements and specifications, including but not limited to FDA, GLP, GMP and ISO standards, rules, requirements and specifications.

The pH of the final compositions of the present invention, for example the “D2 formulation,” is preferably kept at a pH of from about 7.1 to about 7.3. It is also contemplated that there may be some variations in the pH of the final compositions of the present invention, and such variations are also within the scope of the invention.

Moreover, the osmolality of the final compositions of the present invention, for example the “D2 formulation,” is preferably kept within a range of from about 320 mM/Kg to about 430 mM/Kg. It is also contemplated that there may be some variations in the osmolality of the final compositions of the present invention, and such variations are also within the scope of the invention.

For measurements of sterility of the final compositions of the present invention, for example the Second Trace formulation, both aerobic growth and anaerobic growth can be measured.

### Applications and Uses of the Compositions and Formulations of the Present Invention

For all of the representative applications and uses of the inventive compositions and formulations described herein, it is to be understood that the organs and tissues to be preserved by perfusion and/or contact with the compositions and formulations of the present invention include, but are not limited to, kidney, liver, lung, heart, heart-lung in combination, pancreas, and other organs of the digestive tract, blood vessels, endocrine organs or tissue, skin, bone, and other organs and tissues too numerous to mention.

For all of the representative applications and uses of the inventive compositions and formulations described herein, it is also to be understood that the compositions and formulations of the present invention can be used for both civilian and military humans, as well as any type of non-human animal (including any type of mammal or non-mammalian animal). Representative examples of non-human animals include, but are not limited to, dogs, cats, horses, pigs, sheep, rabbits, mice, rats, etc.

Moreover, the present invention contemplates that the compositions and formulations described herein can be administered via any suitable route of administration including, but not limited to, intravenous delivery or by other suitable means for perfusion. For example, in animal studies (such as, but not limited to, safety studies in canines), the compositions and formulations described herein can be administered as an intravenous fluid.

As described herein, conventional approaches for organ preservation and storage (wherein the term “conventional approaches” is intended to refer to previous flawed approaches by others) typically utilize hypothermic conditions for preserving an organ, and their conventional approaches are typically combined with a reduction in organ temperature (e.g., to just above the freezing point of water) to reduce the metabolic rate of organ tissues. However, these conventional approaches lead to damage to organs, tissue, embryos, sperm and cells, and loss of viability of the organs, tissue, embryos, sperm and cells. The novel compositions and formulations of the present invention overcome these serious drawbacks and disadvantages, because the novel compositions and formulations of the present invention can be used as an organ preservation medium to preserve one or more organs without having to utilize hypothermic conditions for preserving the one or more organs. The novel compositions and formulations of the present invention thus do not reduce the metabolic rate of organs, tissues, embryos, sperm and cells, and this unexpectedly and significantly enhances the viability of the organs, tissue, embryos, sperm and cells.

Moreover, with regard to the compositions and formulations of the present invention, while certain specific amounts, concentrations, doses or dosage amounts of constituent ingredients have been described herein, it is to be understood that these are non-limiting examples only, and do not limit the scope of the invention in any way. The present invention contemplates that any suitable amount, concentration, dose or dosage amount of the constituent ingredients can be used.

It is also to be understood that for all of the methods described herein, comprising application and use of the novel compositions and formulations of the present invention in a safe and effective manner, the present invention contemplates that such application and use is always closely supervised, regulated and monitored by one or more suitable and approved professionals that have the required professional training. For example, application and use of the novel compositions and formulations of the present invention in a human will always require the close supervision and monitoring by appropriate healthcare professionals.

Applications and Uses as a Blood Substitute, e.g., Perfusion Support for Organs or Tissues Acutely Deprived of Normal Blood Circulation

The invention also includes methods for treating living animals or people in need of such supportive treatment, comprising application and use of the novel compositions and formulations of the present invention in a safe and effective manner. Thus, simply by way of example, the inventive compositions are useful in providing localized or systemic circulatory or perfusion support for organs or tissues acutely deprived of normal blood circulation caused by trauma, e.g., by infusion or temporary circulation of the inventive compositions to support a partially severed limb, or analogous conditions or other traumatic situations, until surgical repair of damaged vasculature is achieved. Thus, for example, the novel compositions and formulations of the present invention can be used as a blood substitute in a safe and effective manner.

The inventive compositions are also contemplated to be employed during or prior to repair of anatomical areas damaged by disease or accident, e.g., aiding in the preservation of a fully or partially severed finger or limb, prior to restoration of circulatory integrity. Such uses and methods also comprise application and use of the novel compositions and formulations of the present invention in a safe and effective manner.

Applications and Uses for Fluid Replacement

The compositions of the invention can be used as a fluid replacement, and in the preservation, storage and transportation of animal organs, tissue, embryos, sperm and cells.

The invention thus also includes methods for treating living animals or people in need of fluid replacement, comprising application and use of the novel compositions and formulations of the present invention in a safe and effective manner. Examples of treating humans or non-human animals in need of fluid replacement include, but are not limited to, treating humans or non-human animals for dehydration.

Preserving and Protecting Intact Tissues and/or Organs that are Intended for Use in Organ Transplantation

The present invention further contemplates methods for preserving and protecting intact tissues and/or organs that are intended for use in organ transplantation, comprising application and use of the novel compositions and formulations of the present invention in a safe and effective manner. Such uses of the novel compositions and formulations of the present invention as an organ preservation medium include, for example, preserving one or more organs when a person/donor has donated one or more organs for use in transplantation to a recipient or patient in need of the one or more organs.

When reference is made to transplantation, e.g., organ transplantation to a donor, it is to be understood that this encompasses transplanted organs from human organ donors, however it is also intended to encompass xenotransplantation. By using the inventive compositions, e.g. by perfusing organs that are stored before transplantation, organs may be preserved for sustained periods of time. For example, for illustration only, organs be stored from about 48 hours to about 72 hours under suitable and appropriate storage temperatures (e.g., from about 2° Celsius to about 8° Celsius).

The invention also includes methods of treating or supporting tissues or organs in an animal or person after clinical death has occurred, but before the organ or tissue of interest has been removed for donation, comprising application and use of the novel compositions and formulations of the present invention in a safe and effective manner. Any organs that require osmotic and nutritional support for optimal storage and transport benefit from the inventive compositions, both in vivo and in vitro.

The organs and tissues to be preserved by perfusion and/or contact with the inventive compositions and formulations include, but are not limited to, kidney, liver, lung, heart, heart-lung in combination, pancreas, and other organs of the digestive tract, blood vessels, endocrine organs or tissue, skin, bone, and other organs and tissues too numerous to mention.

Preserving and Protecting Tissues and/or Organs During Surgical Procedures

The present invention further contemplates methods for preserving and protecting tissues and/or organs during surgical procedures, e.g., in situations where local blood circulation is interrupted or compromised, comprising application and use of the novel compositions and formulations of the present invention in a safe and effective manner. Such situations include, for example, perfusion of tissues or organ(s) as part of a surgical procedure requiring local or systemic circulatory interruption.

Preserving and Protecting Tissues and/or Organs for Research and/or Diagnostic Purposes

It is further contemplated that the inventive compositions are useful in preserving living and static biological for both humans and animals in research settings where viable cell, organ and other culture techniques are needed for basic and applied biomedical research and/or diagnostic procedures requiring preserving tissue viability in vitro. Such methods for preserving this biological material include application and use of the novel compositions and formulations of the present invention in a safe and effective manner. One example of use in a research setting is the preservation of guinea pig isolated hearts perfused at low flow with the D2 formulation at room temperature. The compositions can also be used for preservation of other organs such as, for example, kidneys.

The inventive compositions can also be used for perfusing biological material in a safe and effective manner (and which maintains viability of organs, tissues, embryos, sperm or cells, as examples) such that diagnostic procedures and tests can be performed, e.g. for the detection and screening of any pathogens said biological material.

It is further contemplated that the inventive compositions can be used for living and static biological material storage and preservation for shipping for research purposes.

The inventive compositions can be used for both veterinarian applications and human applications, and the compositions can be prepared to meet all FDA regulatory requirements for use in humans. The inventive compositions can also be prepared as a solution or any other suitable form.

### Examples of Other Applications of the Compositions of the Present Invention

In another example, the compositions of the present invention can be used for oxygenated machine perfusion of donor livers or other donor organs. The compositions may also be used for pulsatile perfusion of renal allografts at room temperature.

The compositions can also be used to safely, efficiently and reliably maintain cells and tissues that come out of cryopreservation, for example, in a research setting, such that the cells and tissues have significantly enhanced cell and tissue viability. Conventional or traditional approaches typically utilize a very high percentage of dimethyl sulfoxide (DMSO), e.g. between about 5% to about 10% of DMSO, which can be very damaging to cells and tissues and have adverse consequences. In contrast, according to the present invention, it has surprisingly been found that significantly less DMSO is required (e.g., only from about 2% DMSO to about 5% DMSO is needed) when methods are utilized to safely, efficiently and reliably maintain cells and tissues that come out of cryopreservation.

Yet another example is use of the compositions as an organ preservation solution for ex vivo lung perfusion and transplantation, and to protect pulmonary microvasculature. Yet another non-limiting example is use of the compositions to provide a transport system for mouse epididymal sperm.

Yet another non-limiting example is use of the compositions for preservation of motility and fertilization potential in thawed cryopreserved mouse sperm.

The present invention also provides for use of the compositions in oxygen-enriched medium for tumor tissue transport.

The present invention also provides for use of the compositions for maintaining xeno-free human feeder cells for human embryonic stem cell culture.

It has also been surprisingly found that the compositions of the invention, in addition to being an organ preservation solution, also protect pulmonary microvasculature during ex vivo lung perfusion and transplantation.

### EXAMPLES

A number of examples are described and presented below in order to illustrate the present invention, without limiting the scope of the present invention in any way.

### Example 1: Preparation of the “LQL09 WI REV00” “D2 Formulation”

In this representative example, using the ingredients or constituent chemicals described below, the preferred composition of the present invention (hereinafter referred to as the “D2 Formulation”) is prepared. The D2 Formulation includes the following components:
The First Trace$
The Second Trace
The Base$
Sodium Hydroxide (NaOH)
L-Cystine; and
L-Tyrosine.

Summary of Preparation of the Ingredients/Components for the D2 Formulation:

(i): Preparation of the First Trace solution: the following chemicals are dissolved one at a time in 10-35 mL of Absolute Ethanol. Then the solution is brought to a final volume with deionized (DI) water. *Chemicals are measured in μL (with a pipette). **Measure in mL (with a pipette). This First Trace solution is preferably stored at −20 to −5° C.

Record Weights, Scale ID, and Initial each chemical as per formulation,
placing each chemical in the vat.

| CN-Number | Chemical | mg, μL, mL/L | g, μL, mL/Lot |
|---|---|---|---|
| CN-E002 | Ethanol, Absolute | Record | |
| | (C6H6O) | | |
| | (measure in mL) | | |
| CN-A056 | Arachidonic Acid | 2.00 | 0.200 |
| | (C20H32O2) | | |
| *CN-L008 | Linoleic Acid | 10.00 | 1000.00 |
| | (C18H32O2) | | |
| | measure in μL | | |
| CN-L017 | Linolenic Acid | 10.00 | 1.000 |
| | (C18H30O2) | | |
| CN-M033 | Myristic Acid | 10.00 | 1.000 |
| | (CH3(CH2)12COOH) | | |
| *CN-O003 | Oleic Acid (C18H34O2) | 10.00 | 1000.00 |
| | measure in μL | | |
| CN-P065 | Palnnitic Acid. Sodium | 10.00 | 1.000 |
| | (C16H31NaO2) | | |
| CN-S045 | Stearic Acid | 10.00 | 1.000 |
| CN-C046 | Cholesterol | 220.00 | 22.000 |
| **CN-T051 | Tween 80 | 2.20 | 220.00 |
| | (Polysorbate 80) | | |
| | non-animal origin | | |
| | measure in mL | | |
| CN-T036 | DL-α-Tocopherol | 70.00 | 7000.00 |
| | measure in μL | | |
| CN-V003 | Vitamin A Acetate | 3.000 | 0.300 |
| | (Retinol) (C22H32O2) | | |
| | | Total: 357.200 | Total: 9246.500 |

(ii) Preparation of the Second Trace chemicals: the ingredients for the Second Trace chemicals are ground in a mortar and pestle until it is a fine homogenous powder. (CN-0051 may be substituted with CN-0010 only if the lot has been verified from a non-animal source). A lot size of 1000 L (liters) is made. The Second Trace powder is preferably stored at a temperature of from about 2° C. to about 8° C.

Record Weights, Scale ID, and Initial each chemical as per formulation,
placing each chemical in the vat.

| CN-Number | Chemical | mg/L | g/Lot |
|---|---|---|---|
| CN-B002 | D-Biotin | 0.020 | 0.020 |
| CN-C051 | L-Cysteine Hydrochloride | 40.00 | 40.00 |
| | Monohydrate (Non-Animal) | | |
| CN-F009 | Folic Acid | 0.400 | 0.400 |
| CN-G015 | Glutathione, Reduced | 10.00 | 10.00 |
| CN-R001 | Riboflavin | 2.00 | 2.000 |
| CN-T009 | Thiamine, hydrochloride | 10.00 | 10.00 |
| CN-V004 | Vitamin B12 | 0.200 | 0.200 |
| | | Total: 62.620 | Total: 62.62 |

(iii) Preparation of the Base powder: the following chemicals are mixed. It is preferred that the chemicals are milled in short intervals of about 1 hour to prevent sticking to the mill. Then the mill is allowed to sit for about 30 minutes between intervals. In this example, a lot size of 500 liters (500 L) is prepared.

Record Weights, Scale ID, and Initial each chemical as per formulation,
placing each chemical in the vat.

| CN-Number | Chemical | mg/L | g/Lot |
|---|---|---|---|
| CN-A011 | L-Arginine, | 75.00 | 37.50 |
| | Hydrochloride | | |
| CN-A013 | L-Aspartic Acid | 60.00 | 30.00 |
| CN-A040 | Adenosine | 1000.00 | 500.00 |
| CN-A028 | L-Ascorbic Acid | 6.00 | 3.000 |
| CN-C021 | Calcium Chloride, | 120.00 | 60.00 |
| | anhydrous | | |
| CN-C037 | Choline Bitartrate | 453.52 | 226.76 |
| CN-D016 | Dextran-40 | 50000.00 | 25000.00 |
| CN-G007 | Glycine | 50.000 | 25.00 |
| CN-G009 | L-Glutamic Acid | 150.000 | 75.00 |
| CN-G010 | L-Glutamine | 350.00 | 175.00 |
| CN-G013 | D-Glucose, anhydrous | 5000.00 | 2500.00 |
| CN-H002 | L-Histidine, | 164.00 | 82.00 |
| | hydrochloride, | | |
| | monohydrate | | |
| CN-I003 | L-Isoleucine | 25.00 | 12.50 |
| CN-L005 | L-Leucine | 50.00 | 25.00 |
| CN-L006 | L-Lysine, | 240.00 | 120.00 |
| | Hydrochloride | | |
| CN-M022 | Magnesium Sulfate, | 1130.00 | 565.00 |
| | Anhydrous | | |
| CN-M027 | D-Mannose | 10000.00 | 5000.00 |
| CN-P015 | L-Proline | 50.00 | 25.00 |
| CN-P028 | L-Phenylalanine | 50.00 | 25.00 |
| CN-P031 | Potassium Phosphate | 3300.00 | 1650.00 |
| | Monobasic | | |
| CN-P053 | Poloxamer 188/Pluronic | 1000.00 | 500.00 |
| | F-68 (C3H60•C2H40)x | | |
| CN-S024 | Sodium Phosphate, | 300.00 | 150.00 |
| | monobasic, | | |
| | monohydrate | | |
| CN-S044 | Sodium Gluconate | 21000.00 | 10500.00 |
| CN-T002 | L-Threonine | 75.00 | 37.50 |
| CN-T008 | L-Tryptophan | 40.00 | 20.00 |
| CN-V001 | L-Valine | 65.00 | 32.50 |
| | | Total: 94753.52 | Total: 47376.76 |

In this example, this lot of the Base was formulated accurately and in accordance with all current standard operating procedures pertaining to powder media production. All the lots also undergo rigorous milling and process quality testing. Particle size testing is also performed. The Base is preferably stored at a temperature of from about 2° C. to about 8° C.

### Sample Test Results for the Base:

| Test | Specifications |
|---|---|
| Particle size | 90% (through 100 Mesh) |
| Expiration | 48 Months |

(iv) Preparation of the final “D2 Formulation”: using the ingredients as described above, the final “D2 Formulation” is prepared according to the following representative procedure. As such, the following components are combined:
The First Trace solution$
The Second Trace chemicals
The Base powder$
Sodium Hydroxide (NaOH)
L-Cystine; and
L-Tyrosine.

In this non-limiting example, a 100 liter (100 L) lot size is prepared, and is preferably stored at a temperature of from about 2° C. to about 8° C. Also, in this non-limiting example, during the preparation, the following step is performed: *Approximately 1 to 2 g of *CN-S004 (Sodium Hydroxide) is dissolved in about 100 mL water. Then *CN-0018 (L-Cystine) and *CN-T004 (L-Tyrosine) are dissolved in the *CN-S004 solution. (More *CN-S004 is added if needed to solubilize.) The *CN-S004 solution [containing the *CN-0018 (L-Cystine) and *CN-T004 (L-Tyrosine) are dissolved in the *CN-S004 solution] is then added to the vat comprising the First Trace solution, xli Second Trace chemicals, and the Base powder.

Record Weights, Scale ID, and Initial each chemical as per formulation,
placing each chemical in the vat.

| CN-Number | Chemical | mg, mL/L | g, mL/Lot |
|---|---|---|---|
| LQL02 | First Trace solution | 10.00 | 1000.00 |
| TRACE 1 | measure in mL | | |
| LQL09 | Second Trace | 62.620 | 6.262 |
| TRACE 1 | chemicals | | |
| LQL02 BASE | Base | 94753.52 | 9475.35 |
| *CN-S004 | Sodium Hydroxide | Record | |
| | (NaOH) | | |
| *CN-C018 | L-Cystine | 19.55 | 1.955 |
| *CN-T004 | L-Tyrosine | 57.70 | 5.770 |
| | | Total: 94903.390 | Total: 10489.34 |

The pH of the “D2 Formulation” is preferably kept at a pH of from about 7.1 to about 7.3.

The Osmolality of the “D2 Formulation” is preferably kept within a range of from about 320 mM/Kg to about 430 mM/Kg.

In this Example, Quality Control Studies of The “D2 Formulation Yielded the Following Results:

| Test | Specifications |
|---|---|
| Appearance | Clear, Yellow Solution |
| Sterility | Pass |
| pH | 7.1-7.3 |
| Osmolality | 320-430 mM/Kg |
| Endotoxin | ≤5.0 EU/mL |
| Mycoplasma | Negative |
| Expiration | 12 Months |

For measurements of sterility of the “D2 Formulation, both Aerobic Growth and Anaerobic Growth can be measured.

### Preliminary Heart Transplant Results (Cervical Model)

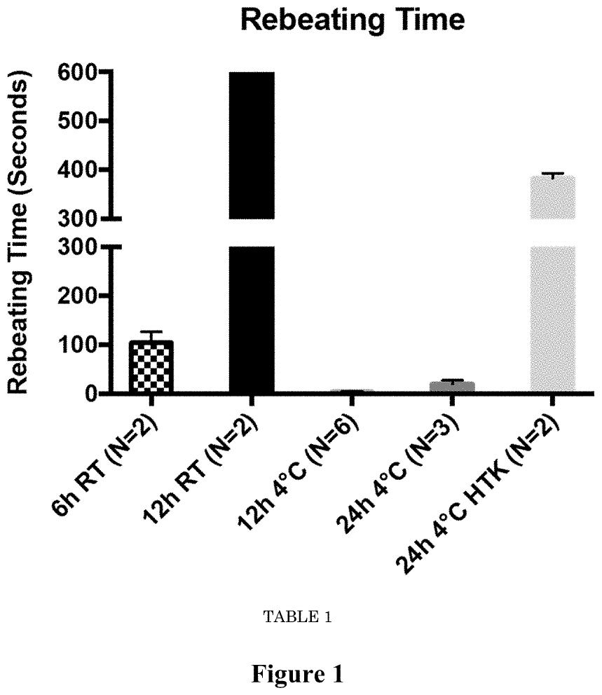

Turning now to FIG. 1, a cervical model heart transplant study (allotransplantation study) completed at John's Hopkins University depicts the re-beating time in seconds (s) after a heart graft re-transplant in mice. In the first study done at room temperature, the heart was isolated from the mice (n=2) and stored in the D2 solution (the present invention) for 6 hours (t=6 h) at room temperature (37° C.). Once transplanted, the heart initiated its beating within 100 sec (1 minute, 40 sec). The re-beating time prolonged for more than 600 sec (10 min), though the heart transplant proved unsuccessful.

Thereafter, the D2 solution was compared against HTK (histidine-tryptophan-mice (n=6) and stored at 4° C. for 12 hours. When the re-graft was initiated, beating began within less than 10 seconds while hearts isolated from 3 mice (n=3) stored at 4° C. under similar conditions to the first trial for 24 hours. When re-transplanted the heart initiated to beat at less than 30 seconds. With similar conditions of hypothermia, the heart was removed from 2 mice (n=2) and preserved in HTK at 4° C. for 24 hours and re-transplanted. Compared to the treatment of the graft with the D2 solution, 24-hour hypothermic preservation using HTK prolonged the time required for the heart to re-beat after transplantation, an undesirable result for HTK. While hearts treated with the D2 solution at 24-hour preservation when re-grafted initiated the double circulation within a span of less than 50 seconds, and hearts preserved in HTK for the same time period when re-grafted took more than 300-600 seconds (6-10 min) to initiate filling of the heart and recruit blood for circulation, thereby demonstrating that the D2 of the present invention is a vast improvement over HTK. Hypothermic preservation of mice heart using D2 treated grafts reduced the re-beating time by 60-fold as compared to HTK.

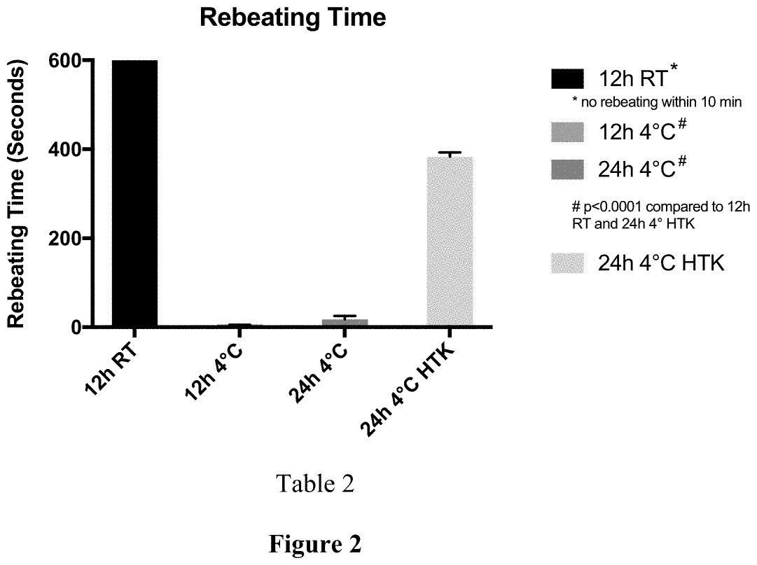

FIG. 2 establishes a circulatory relationship between hypothermic preservation at 12 h and 24 h. When statistically compared the relationship between re-beating and the time required for the grafts to beat after transplant, a statistically significant difference (p<0.0001) was identified at preservation times 12 and 24 hours. This elaborates that time of preservation has an effect on initiating the beating of the heart after allotransplantation.

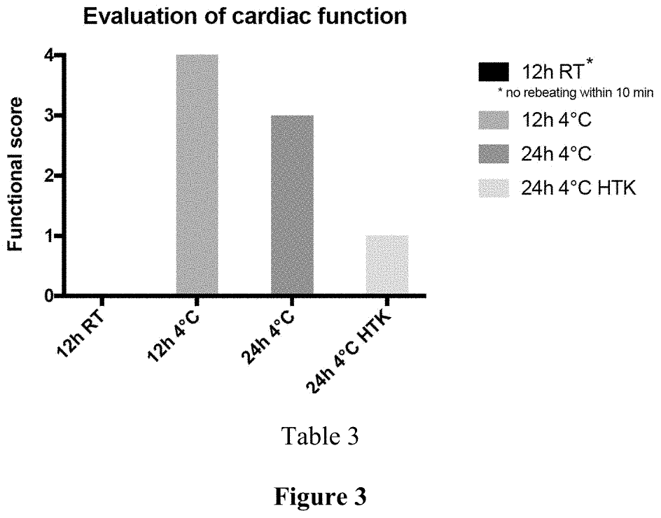

FIG. 3 is an evaluation of cardiac function wherein its functional score is performed to identify cardiac function after transplantation. A study at room temperature preservation with D2 for 12 hours provided no functional score as re-beating was not established even after 10 minutes. Hence no evaluation was performed. Study with D2 for room temperature preservation was performed but a cardiac functional score was not evaluated at this stage, though beating of the heart was noted after 100 seconds (see FIG. 1). However, when a study of hypothermic preservation using D2 against HTK, the cardiac functional score during hypothermic preservation at 12 and 24 hours using D2 had a much higher score as compared to 24-hour preservation with HTK. Comparison between 12 and 24-hour preservation with D2 has shown that 12-hour preservation has a significantly higher score than 24 hours. Comparing it to HTK at 24-hour preservation, the functional score of cardiac function at 24 hours using D2 is 3-fold higher than HTK, and at 12 hours D2 is higher than HTK by 4-fold. In conclusion, at 12 hours and 24 hours cardiac function is equally normal for the heart treated with D2 under hypothermic conditions, while hypothermic preservation using HTK significantly lowered the cardiac function of the transplanted hearts. And so, D2 of the present invention is able to preserve hearts both at room temperature and at hypothermic temperatures in a better state than HTK, which is one of the most currently used, and state of the art, solutions in preservation of mammalian hearts.

### Preliminary Heart Transplant Results (Abdominal Model)

TABLE 4

| | Surgical | POD 0 | | |
|---|---|---|---|---|
| | attempt | monitor | Follow-up | Harvest |
| 6 h RT | 1 | No re-beat | Graft recovered | 1/1 |
| | | | on POD1, beating | |
| | | | is very weak. | |
| 24 h 4° C. | 1 | Very delayed | Graft recovered | 1/1 |
| | | re-beat (over | on POD1, beating | |
| | | 6 min until | is weak. | |
| | | beating) | | |
| 36 h 4° C. | 3 | Very delayed | On-going | 1/3 |
| | | re-beat (over | | |
| | | 6 min until | | |
| | | beating) and | | |
| | | rebeating was | | |
| | | weak. | | |

Table 4 depicts an abdominal model heart transplant study in mice completed at John's Hopkins University on postoperative day zero (POD 0) for establishing the re-beating time at room temperature after 6 hours and at 4° C. after 24 and 36 hours, respectively, as well on postoperative day 1 (POD 1) for establishing the beating strength of the heart graft when recovered at room temperature after 6 hours and at 4° C. after 24 and 36 hours, respectively, using the novel solution D2 of the present invention.

As shown therein, no re-beat was established on POD 0 at room temperature after 6 hours and the re-beat was very weak on POD 1 when the heart graft was recovered. In contrast, at 4° C. after 24 hours on POD 0, beating was delayed but it was re-established at just over 6 minutes, and on POD 1 the heart graft recovered immitted a weak beat. And, at 4° C. after 36 hours on POD 0, beating was delayed but it was re-established at just over 6 minutes, albeit a weak beat, and on POD 1, the follow up was still ongoing.

1. A composition comprising a first trace, a second trace and a base,
wherein the first trace comprises Arachidonic Acid, Linoleic Acid, Linolenic Acid, Myristic Acid, Oleic Acid, Palmitic Acid Sodium, Stearic Acid, Cholesterol, Tween80 (Polysorbate 80), DL-a-Tocopherol and Vitamin A acetate (Retinol),
wherein the second trace comprises D-Biotin, L-Cysteine hydrochloride monohydrate, Folic Acid, Reduced Glutathione, Riboflavin, Thiamine hydrochloride and Vitamin B12,
wherein the base comprises L-Arginine hydrochloride, L-Aspartic Acid, Adenosine, L-Ascorbic Acid, Calcium Chloride anhydrous, Choline Bitartrate, Dextran-40, Glycine, L-Glutamic Acid, L-Glutamine, D-Glucose anhydrous, L-Histidine hydrochloride, monohydrate, L-Isoleucine, L-Leucine, L-Lysine hydrochloride, Magnesium Sulfate anhydrous, D-Mannose, L-Proline, L-Phenylalanine, Potassium Phosphate monobasic, Poloxamer 188/Pluronic F-68, Sodium Phosphate monobasic monohydrate, Sodium Gluconate, L-Threonine, L-Tryptophan and L-Valine, and further wherein the composition comprises L-Cystine and L-Tyrosine.

2. The composition of claim 1, wherein the composition is a solution.

3. The composition of claim 1, wherein the composition is essentially free of all human and non-human animal proteins.

4. The composition of claim 1, wherein the composition comprises at least one nanoparticle or liposome component.

5. The composition of claim 1, wherein the composition is essentially free of any viruses or other pathogens.

6. The composition of claim 2, wherein the pH of the composition is from about 7.1 to about 7.3.

7. The composition of claim 2, wherein the osmolality of the composition is within a range of from about 320 mM/Kg to about 430 mM/Kg.

8. A method of preparing the composition of claim 1, comprising combining the first trace, the second trace and the base.

9. The method of claim 8, wherein the pH of the composition is from about 7.1 to about 7.3.

10. The method of claim 8, wherein the composition is essentially free of all human and non-human animal proteins.

11. A method of preserving a mammalian organ, ex vivo, comprising contacting or perfusing the mammalian organ with an effective amount of the composition of claim 2.

12. A method of providing perfusion support for an organ or tissue acutely deprived of normal blood circulation, comprising administering the composition of claim 2 to the organ or tissue.

13. A method of treating a human or non-human animal in need of fluid replacement, comprising administering the composition of claim 2 to the human or non-human animal in need thereof.

14. A method of protecting an organ, tissue, or cell, comprising administering the composition of claim 1 to the organ, tissue or cell in need thereof.

15. A method of repairing an anatomical area damaged by disease or accident, comprising administering the composition of claim 1 to the anatomical area in need thereof.

16. A composition comprising Arachidonic Acid, Linoleic Acid, Linolenic Acid, Myristic Acid, Oleic Acid, Palmitic Acid Sodium, Stearic Acid, Cholesterol, Tween80 (Polysorbate 80), DL-a-Tocopherol, Vitamin A acetate (Retinol), D-Biotin, L-Cysteine hydrochloride monohydrate, Folic Acid, Reduced Glutathione, Riboflavin, Thiamine hydrochloride, and Vitamin B12, L-Arginine Hydrochloride, L-Aspartic Acid, Adenosine, L-Ascorbic Acid, Calcium Chloride anhydrous, Choline Bitartrate, Dextran-40, Glycine, L-Glutamic Acid, L-Glutamine, D-Glucose anhydrous, L-Histidine hydrochloride monohydrate, L-Isoleucine, L-Leucine, L-Lysine hydrochloride, Magnesium Sulfate (anhydrous), D-Mannose, L-Proline, L-Phenylalanine, Potassium Phosphate monobasic, Poloxamer 188/Pluronic F-68, Sodium Phosphate monobasic monohydrate, Sodium Gluconate, L-Threonine, L-Tryptophan and L-Valine, L-Cystine and L-Tyrosine.

17. A system for maintaining the viability of living and static biological material, wherein:
a) the system comprises the composition of claim 1,
b) the system protects, maintain, and enhances the viability of the living and static biological material; and
c) the composition optionally comprises at least one nanoparticle or liposome component.

18. The system of claim 17, wherein the living and static biological material is chosen from the group consisting of organs, tissue, embryos, sperm and cells.

19. The system of claim 18, wherein the biological material is chosen from human and non-human mammals.

20. The system of claim 17, wherein the composition is essentially free of all human and non-human animal proteins, or essentially free of any viruses or other pathogens.
